(19) United States
(12) Patent Application Publication
(10) Pub. No.: US 2022/0127646 A1
(43) Pub. Date: Apr. 28, 2022

(54) METHODS AND APPARATUS FOR HYDROGEN BASED BIOGAS UPGRADING

(71) Applicant: AgorFora ApS, Korsor (DK)

(72) Inventors: Irini ANGELIDAKI, Frederiksberg (DK); Poul LYHNE, Holstebro (DK); Gang LUO, Lyngby (DK)

(21) Appl. No.: 17/543,090

(22) Filed: Dec. 6, 2021

Related U.S. Application Data
(63) Continuation of application No. 14/353,299, filed on Aug. 4, 2014, now Pat. No. 11,193,142.
(60) Provisional application No. 61/550,492, filed on Oct. 24, 2011.
(60) Provisional application No. 61/563,247, filed on Nov. 23, 2011.

(30) Foreign Application Priority Data
Oct. 24, 2011 (EP) EP11186323.9

(51) Int. Cl. C12P 5/02; C12M 1/00; C12M 1/107

(52) U.S. Cl. CPC C12P 5/023; C12M 29/16; C12M 21/04; Y02E 50/30; C12M 43/00; C12M 47/18; C12M 43/08

(57) ABSTRACT

The present invention relates to an anaerobic process for biogas upgrading and hydrogen utilization comprising the use of acidic waste as co-substrate. In this process, H₂ and CO₂ will be converted to CH₄, which will result in lower CO₂ content in the biogas. The invention relates to both in situ and ex situ methods of biogas upgrading. The invention further relates to a bioreactor comprising hollow fiber membranes.

### CROSS REFERENCE TO RELATED APPLICATIONS

The present application is a Continuation of U.S. patent application Ser. No. 14/353,299, filed Apr. 22, 2014, now U.S. Pat. No. 11,193,142, issued Dec. 7, 2021, which is a National Stage application under 35 U.S.C. § 371, of PCT/DK2012/050390, filed Oct. 12, 2012, which claims benefit of priority from U.S. Patent Provisional Application No. 61/550,492, filed Oct. 24, 2011, and which claims benefit of priority from U.S. Patent Provisional Application No. 61/563,247, filed Nov. 23, 2011, the entirety of which are expressly incorporated herein by reference. The present application also claims benefit of priority from European Patent Application EP11186323.9, filed Oct. 24, 2011.

### FIELD OF INVENTION

The present invention relates to the field of biogas upgrading in anaerobic biogas reactors by hydrogen utilization.

### BACKGROUND OF INVENTION

The production and utilization of biogas by anaerobic digestion of organic wastes is an emerging alternative energy technology. Biogas is envisioned as a key element in emerging renewable energy strategies in Europe, motivated by the European Union target of achieving 20% renewable energy by 2020. The Danish government also proposed a target of using 50% of the manure produced in Denmark for renewable energy production by 2020, and it would essentially be met through a strong biogas expansion. Biogas mainly contains CH₄ (50-75%) and CO₂ (25-50%). Upgrading of biogas to CH₄ content higher than 90% can not only increase the heating value, but also reduce corrosion caused by acid gas and therefore extend the biogas utilization as a renewable energy source.

The common methods of biogas upgrading include water washing, pressure swing adsorption, polyglycol adsorption and chemical treatment, which aim to remove CO₂ from the biogas. The costs of the above methods are relatively high since they need either high pressure or addition of chemicals. Besides, when removing CO₂ from biogas, small amounts of CH₄ are also removed, which will possibly increase the greenhouse gas emission. To circumvent these disadvantages and use milder treatments with minimal chemical and energy, anaerobic microorganisms can be used to convert CO₂ to CH₄ for biogas upgrading (equation 1). The microorganisms are hydrogenotrophic methanogens within the orders Methanobacteriales, Methanococcales, Methanomicrobials et al. Anaerobic sludges in anaerobic reactors treating organic wastes contain the above microorganisms and have certain hydrogenotrophic-methanogenic potential. Therefore, hydrogen can be injected into an anaerobic reactor for in-situ CO₂ reduction and biogas upgrading.

4H₂+CO₂=CH₄+2H₂O ΔG⁰=−130.7 KJ/mol (1)

The H₂ used for CH₄ production from CO₂ can be obtained from wind mills.

Commercial wind power has been produced in Denmark since 1970s, and wind power currently accounts for nearly 20% of the Danish electricity supply. Due to varying wind conditions and electricity demand over the year, up to 40% of the electricity from wind is judged to be surplus. An attractive way of exploiting wind mills capacity is to electrolyze water for H₂ production. In addition, H₂ can be also obtained by other sources, including coal gasification, petroleum refinery, petrochemical plants, and soda manufacture. Currently, hydrogen utilization has several yet unsolved bottlenecks such as hydrogen transport, storage, as well as utilization in the fuel cells. Therefore, conversion of hydrogen in a biogas plant would provide several advantages, such as utilization of the existing infrastructure of biogas plants. Additionally, conversion of hydrogen in a biogas reactor would consume some CO₂ in the biogas and thereby result in upgraded biogas with lower CO₂ content. This partial removal of CO₂ from the biogas would decrease the costs for the upgrading of biogas to natural gas quality, which could provide additional utilization opportunities of biogas, e.g., as vehicle fuel, as well as improving energy density and transmission capacity of the CH₄ enriched biogas. Finally, possible unconverted hydrogen mixed with methane, would improve the combustion properties of biogas as fuel (5-30% hydrogen by volume). The storage cost for methane is at least a 3 times lower compared to hydrogen, due to the higher boiling point and higher volumetric energy density of gaseous methane. Additionally, a number of countries already have natural gas infrastructure, which would make distribution of upgraded biogas feasible.

US 2009/013734 discloses a method for converting effluent CO₂ (g) from ethanol plants and H₂ (g) to CH₄ (g). The H₂ (g) is typically produced by hydrolysis using inexpensive off-peak electricity from power plants. The process is performed by microbial fermentation using methanogenic archaea.

### SUMMARY OF INVENTION

Biogas is currently one of the fastest growing sources of energy. However, biogas is produced from biomass and contains a high proportion of CO₂ which has a low value as energy source. Thus, it is desirable to decrease the content of CO₂ in biogas. This process, which often is referred to as upgrading of biogas, is facilitated by methanogenic organisms, preferably hydrogenotrophic methanogenic archaea. These organisms are preferably cultured at a pH of between 7-8. The process includes adding H₂ containing gas or pure H₂ (g) to a biogas reactor for in-situ biogas upgrading. The hydrogenotrophic methanogenic archaea utilizes the H₂ to convert CO₂ of the biogas and/or CO₂ dissolved in the culture medium to CH₄.

However, as the process progresses, the CO₂ of the culture medium is consumed resulting in an increased pH of the culture medium. When pH exceeds 8.3 the process is inhibited as the culturing conditions for the methanogenic archaea has become suboptimal. In order to solve this problem, the present inventors have demonstrated that the anaerobic digestion of the biomass, such as manure can be made in the presence of an acidic composition such as acidic waste from industry and/or agriculture, resulting in a sustained and high-performing process for upgrading biogas. The upgraded biogas can be utilized in existing gas grid infrastructure.

According to the present invention, the biogas upgrading may be performed in a single bioreactor, i.e., in situ, or in separate bioreactors, i.e., ex situ.

In one aspect the present invention relates to a method of manufacturing upgraded biogas in situ, said method comprising the steps of:

a. initiating an anaerobic digestion process in a bioreactor comprising:
i. substrate,
   ii. anaerobic inoculum comprising anaerobic hydrogenotrophic methanogenic organisms
b. feeding the bioreactor with acidic waste having a pH<5,
c. feeding the bioreactor with biomass,
d. injecting H₂ containing gas into the bioreactor, and
e. collecting the upgraded biogas thus produced, wherein said upgraded biogas has a CH₄ content of at least 90%.

In one aspect the present invention relates to a method of manufacturing an upgraded biogas, wherein said biogas is further upgraded in a second bioreactor (ex situ) according to a method comprising the steps of:

a. initiating an anaerobic digestion process in a first bioreactor comprising:
i. substrate,
ii. anaerobic inoculum comprising anaerobic hydrogenotrophic methanogenic organisms,

b. feeding the first bioreactor with acidic waste having a pH<5.

c. feeding the bioreactor with biomass,

d. injecting H₂ containing gas into the first bioreactor,

e. transferring the biogas produced in the first bioreactor to a second bioreactor, wherein an anaerobic digestion process has been initiated with: nutrients, anaerobic inoculum comprising anaerobic hydrogenotrophic methanogenic organisms, feeding the second bioreactor with nutrients,

g. injecting H₂ containing gas into the second bioreactor, and

h. collecting the upgraded biogas thus produced, wherein said upgraded biogas has a CH₄ content of at least 95%.

In another aspect the present invention relates to a bioreactor comprising:

a. reactor vessel,

b. a gas injection system comprising hollow fibers

c. loading means for supply of substrates and or inoculum, and

d. discharge means for products and/or waste.

### DESCRIPTION OF DRAWINGS

FIG. 1: Schematic overview of the plant and process of the invention.

FIG. 2: One embodiment of the plant/method of the present invention.

FIG. 3: Injection system comprising hollow fiber membrane for optimized gas diffusion.

FIG. 4: Schematic ex-situ biogas upgrading reactor.

FIG. 5: Profiles of biogas production, pressure and H₂ flow rate of reactor A.

FIG. 6: Profiles of biogas composition and pH of reactor A.

FIG. 7: Scanning Electron Microscopy (SEM) images of the Hollow Fiber Membrane (HFM): raw HFM (A-C), HFM in the reactor (D-F). Settings were as follows:
A—dwell: 30 μs, HV: 2.00 kV; pressure: 1.05e⁻³ Pa; Spot: 3.0; Mag: 350×
B—dwell: 30 μs, HV: 2.00 kV; pressure: 1.05e⁻³ Pa; Spot: 3.0; Mag: 1,000×
C—dwell: 30 μs, HV: 2.00 kV; pressure: 1.05e⁻³ Pa; Spot: 3.0; Mag: 20,000×
D—dwell: 30 μs, HV: 5.00 kV; pressure: 1.32e⁻³ Pa; Spot: 3.0; Mag: 350×
E—dwell: 30 μs, HV: 5.00 kV; pressure: 1 32e⁻³ Pa; Spot: 3.0; Mag: 1,000×
F—dwell: 30 μs, HV: 5.00 kV; pressure: 1.58e⁻³ Pa; Spot: 3.0; Mag: 20,000×

FIG. 8: H₂ flow rate, pH and bicarbonate in reactor A according to H₂ pressure inside the HFM in each steady state.

FIG. 9: CO₂ in the liquid and gas phase in reactor A in each steady state.

FIG. 10: Configuration of reactor A of Example 3.

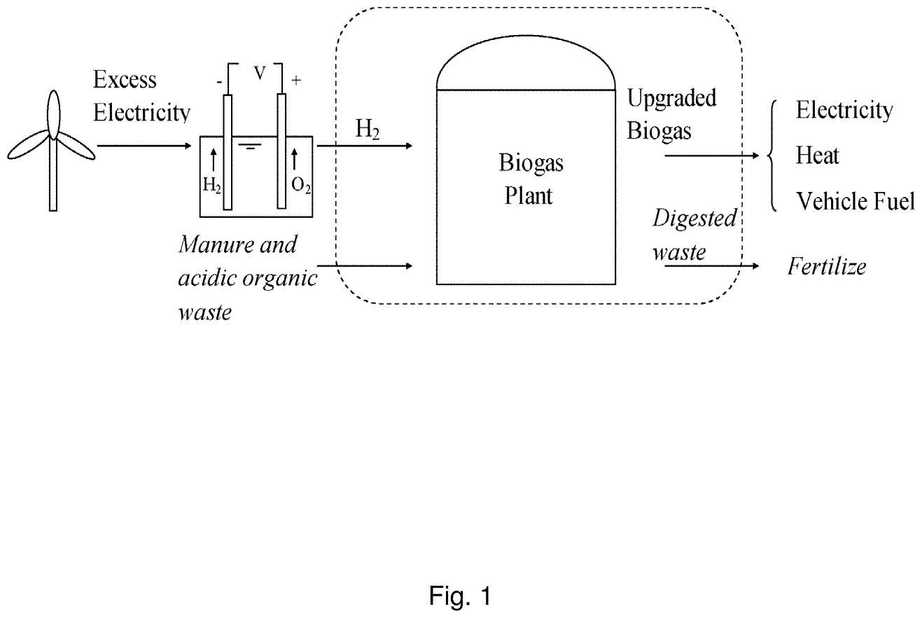

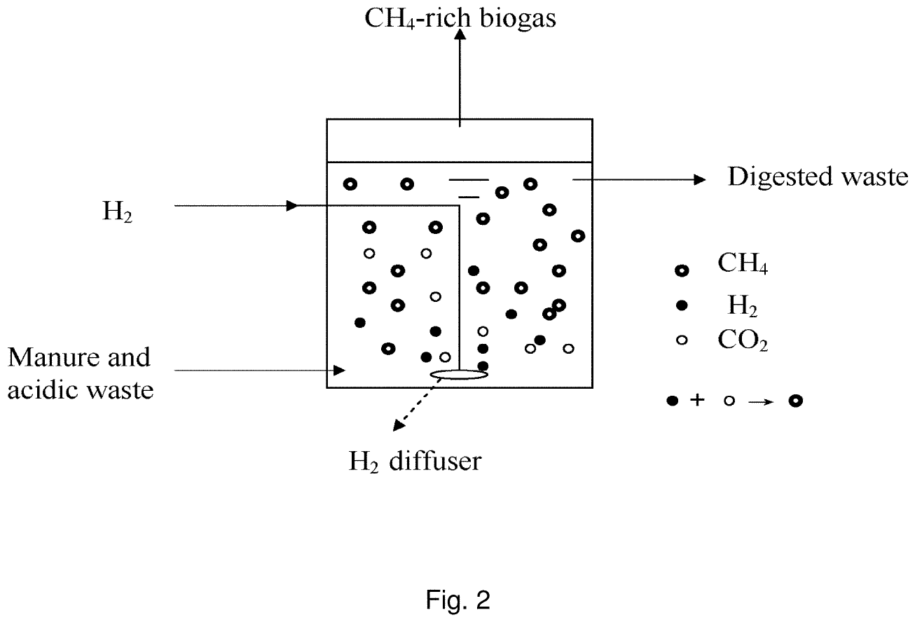

### DETAILED DESCRIPTION OF THE INVENTION

Hydrogen Production and Utilization

Hydrogen (H₂) can be produced by electrolysis of water. To be economically favorable, this requires cheap electric power. The production of electric power in wind power plants varies over time due to wind conditions. The consumption of electric power also varies over time due to circadian rhythm and industrial needs. Hence occasionally the production of energy exceeds the consumption which thus produces surplus energy. The surplus energy can be utilized during off-peak hours to produce cheap H₂ by electrolysis of water.

Attempts to utilize H₂ as energy source in itself are environmentally very promising but at present techniques are not sufficiently efficient and economically favorable. One disadvantage is that hydrogen must be compressed and stored which is expensive.

Biogas

Another sustainable source of energy is biogas which typically is produced during digestion of biomass.

Biogas typically refers to a gas produced by the biological breakdown of organic matter in the absence of oxygen. Organic waste such as manure, dead plant and animal material, animal dung, and kitchen waste can be converted into a gaseous fuel called biogas. Biogas originates from biogenic material and is a type of biofuel.

Biogas is produced by the anaerobic digestion of biodegradable materials (biomass) such as manure, sewage, municipal waste, green waste, plant material, and crops. Biogas comprises primarily methane (CH₄) and carbon dioxide (CO₂) and may have small amounts of hydrogen sulfide (H₂S), moisture and siloxanes.

The gases methane, hydrogen, and carbon monoxide (CO) can be combusted. Energy release through said combustion allows biogas to be used as a fuel. Biogas can be used as a fuel in any country for any heating purpose, such as cooking. It can also be used in anaerobic digesters where it is typically used in a gas engine to convert the energy in the gas into electricity and heat. Biogas can be compressed, much like natural gas, and used to power motor vehicles. In the UK, for example, biogas is estimated to have the potential to replace around 17% of vehicle fuel. Biogas is a renewable fuel, so it qualifies for renewable energy subsidies in some parts of the world. Biogas can also be cleaned and upgraded to natural gas standards when it becomes biomethane.

The composition of biogas varies depending upon the biomass and origin used for the anaerobic digestion process. However, the composition of raw biogas produced by traditional methods typically ranges within the values summarized in table 1 below:

TABLE 1

| Compound | Formula | % |
|---|---|---|
| Methane | CH₄ | 50-75 |
| Carbon dioxide | CO₂ | 25-50 |
| Nitrogen | N₂ | 0-10 |
| Hydrogen | H₂ | 0-1 |
| Hydrogen sulfide | H₂S | 0-3 |
| Oxygen | O₂ | 0-0 |

The composition of biogas varies depending upon the origin of the anaerobic digestion process. Landfill gas typically has methane concentrations around 50%. Advanced waste treatment technologies can produce biogas with 55-75% CH₄, which for reactors with free liquids can be increased to 80-90% methane using in-situ gas purification techniques.

When biogas is used, many advantages arise. In North America, utilization of biogas would generate enough electricity to meet up to three percent of the continent's electricity expenditure. In addition, biogas could potentially help reduce global climate change. Normally, manure that is left to decompose releases two main gases that cause global climate change: nitrous dioxide and methane. Nitrous dioxide (N2O) warms the atmosphere 310 times more than carbon dioxide and methane 21 times more than carbon dioxide. By converting cow manure into methane biogas via anaerobic digestion, the millions of cows in the United States would be able to produce one hundred billion kilowatt hours of electricity, enough to power millions of homes across the United States. In fact, one cow can produce enough manure in one day to generate three kilowatt hours of electricity; only 2.4 kilowatt hours of electricity are needed to power a single one hundred watt light bulb for one day. Furthermore, by converting cow manure into methane biogas instead of letting it decompose, it would be possible to reduce global warming gases by ninety-nine million metric tons or four percent.

The 30 million rural households in China that have biogas digesters enjoy 12 benefits: saving fossil fuels, saving time collecting firewood, protecting forests, using crop residues for animal fodder instead of fuel, saving money, saving cooking time, improving hygienic conditions, producing high-quality fertilizer, enabling local mechanization and electricity production, improving the rural standard of living, and reducing air and water pollution.

In Situ Biogas Upgrading

As mentioned herein above raw biogas produced from digestion in traditional biogas reactors is roughly 60% methane and 40% CO₂. The high CO₂ content makes the biogas less attractive for direct use as energy source. The solution is the use of a biogas upgrading or purification process whereby contaminants in the raw biogas stream are absorbed or scrubbed, leaving up to 98% methane per unit volume of gas. Traditionally there have been four main methods of biogas upgrading including water washing, pressure swing absorption, selexol absorption, and amine gas treating. The most prevalent method is water washing where high pressure gas flows into a column where the carbon dioxide and other trace elements are scrubbed by cascading water running counter-flow to the gas. Present day arrangement, however, are expensive and impractical as they require a further step before the biogas can be utilized. The present inventors have demonstrated that it is possible to obtain large quantities of high-grade (high content CH₄) biogas by in situ utilization of H₂ containing gas or pure H₂ (g).

The inventors have demonstrated that H₂, such as H₂ produced by electrolysis of water using off-peak hours excess electric power, can be utilized directly in a biogas reactor, thus avoiding expensive compression and storage, for upgrading biogas by converting CO₂ in the biogas to methane (CH₄). The upgraded biogas can be utilized in an existing natural gas grid infrastructure and/or to directly as a fuel source.

The process can make use of existing infrastructure of biogas plants and it is therefore a cheap method for upgrading of biogas.

Conversion of CO₂ to CH₄ using H₂ has the following problem—when hydrogen is injected into a bioreactor comprising methanogens, and reacts with CO₂ to produce CH₄, the pH increases due to bicarbonate consumption. When the pH exceeds 8.3, the biological conversion process will be inhibited, because methanogens cannot grow well at this high pH. In order to achieve in situ biogas upgrading and hydrogen utilization with high efficiency, the present inventors have demonstrated that biogas can be produced by co-digestion of manure with acidic wastes. Several acidic wastes can be used as substrates for co-digestion with biomass, such as manure. Examples of acidic wastes include but are not limited to whey wastes, stillage (wastewater from ethanol production) and fruit juice from potato processing industries. The acidic waste thus contributes to keeping the pH in an optimal range of between 7 and 8. Hence the anaerobic process is allowed to proceed without inhibition. A further benefit of the acidic waste is that it can also functions as a substrate for microbial conversion.

The acidic waste usually has a pH lower than about 5.5, such as a pH of about or lower than 5.0, for example a pH of about or lower than 4.5. For example, the pH of whey is about 4 to 5, such as about 4.5.

The acidic waste is in one embodiment mixed with the biomass, such as manure, to create a mixture before being fed to the reactor. This results in a lowering of the pH of the composition which is fed to the reactor. The ratio of acidic waste to biomass is usually about 2:3. However, in other embodiments, the ratio may be about 1:1. The ratio depends on the pH of the acidic waste added. The pH of the mixture of biomass and acidic waste is preferably less than 7.

In another embodiment, the acidic waste is fed to the reactor separately from the biomass. The acidic waste, which may be added together with the biomass or separately from the biomass, may be fed to the reactor once, twice, or several times a day. The essential criterion is that the pH of the reactor is kept in the optimal range of pH 7 to 8 for the anaerobic process to proceed without inhibition. The acidic waste thus acts to stabilize the pH at an optimal level as CO₂ is removed from the biogas which in the absence of acidic compositions such as acidic waste would render the pH to increase.

Accordingly, in one aspect the present invention relates to a method of manufacturing an upgraded biogas, said method comprising the steps of:

a. initiating an anaerobic digestion process in a bioreactor comprising:
i. substrate,
ii. anaerobic inoculum comprising anaerobic hydrogenotrophic methanogenic organisms

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

The substrate is usually biomass comprising the nutrients necessary for the initiation of the anaerobic digestion process; however, the substrate may also consist of nutrients only. The term “nutrients” is to be understood as micro and macro nutrients necessary for microbial growth.

The expression “biomass” as used herein is to be understood as products consisting of organic or vegetable matter from agriculture or forestry, which material can be used as fuel for the extraction of the energy content of the material. Typically, the biomass is protein-rich organic waste, including but not limited to manure, activated sludge from wastewater treatment plant, fish processing residues, etc.

A prerequisite for producing biogas is to have a substrate such as biomass that can be converted to biogas. Biomass is typically used in the initiation of the anaerobic process but is also fed to the bioreactor repeatedly during the biogas production process, such as once, twice, or three times a day. Alternatively, the anaerobic process may be initiated with a minimal nutrients solution only and the biomass is added at a later stage, for example at the same time as the acidic waste.

It is furthermore understood that anaerobic inoculum normally comprises anaerobic hydrogenotrophic methanogenic organisms such as anaearobic hydrogenotrophic archaea. The inoculum may also contain one or more types of microorganisms selected from the group consisting of hydrolytic, acidogenic, acetogenic and methanogenic microorganisms.

Anaerobic inoculum is sometimes also referred to as anaerobic sludge.

The expression high CH₄ content biogas is to be understood as a biogas having a CH₄ content exceeding that of conventional non-upgraded biogas. Hence, according to the present invention, a “high CH₄ content biogas” typically contains at least 90% CH₄, such as at least 91% CH₄, for example at least 92% CH₄, such as at least 93% CH₄, for example at least 94% CH₄, such as at least 95% CH₄, for example at least 96% CH₄, such as at least 97% CH₄, for example at least 98% CH₄, such as at least 99% CH₄, for example at least 99.5% CH₄, such as at least 99.6% CH₄, for example at least 99.7% CH₄, such as at least 99.8% CH₄, for example at least 99.9% CH₄ as measured by CH₄ content of the biogas.

In one embodiment of the method of the present invention the pH in the bioreactor is maintained below 8.3.

In another embodiment of the method of the present invention the pH in the bioreactor is maintained below 8.0.

In one embodiment the pH is maintained between 7 and 8. As mentioned, this can be achieved by the process of co-digesting biomass with acidic waste.

In one embodiment the ratio of biomass to acidic waste is experimentally determined to maintain the pH between 7 and 8.

In one embodiment the ratio of biomass to acidic waste fed to the bioreactor is 3 to 2.

In one embodiment the ratio of biomass to acidic waste fed to the bioreactor is 1 to 1.

In one embodiment the method of the present invention further comprises the step of separating CH₄ (g) from the other components of the high CH₄ content biogas produced, thus producing a substantially pure CH₄ (g).

In one embodiment, the biogas produced by the present invention is further upgraded by traditional methods of upgrading biogas.

In one embodiment, the biogas produced by the present invention is added propane to adjust the burn value of the gas.

In one embodiment the H₂ containing gas is added continuously to the bioreactor.

In one embodiment a gas injection system comprising hollow fibers is used to introduce the H₂ into the bioreactor.

The method of manufacturing an upgraded biogas comprises the process of increasing the CH₄ content of the biogas, thus in one aspect the present invention relates to a method for increasing CH₄ content in biogas in situ, said method comprising the steps of:

a. initiating an anaerobic digestion process in a bioreactor comprising:
i. substrate,
ii. anaerobic inoculum comprising anaerobic hydrogenotrophic methanogenic organisms

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

Likewise, the method of manufacturing an upgraded biogas comprises the process of decreasing the CO₂ content of the biogas, hence in one aspect the present invention relates to a method for reducing CO₂ (g) content in biogas, said method comprising the steps of:

a. initiating an anaerobic digestion process in a bioreactor comprising:
i. substrate,
ii. anaerobic inoculum comprising anaerobic hydrogenotrophic methanogenic organisms

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

By removing the low energy content CO₂ from the biogas, the overall energy content of the biogas is increased. Hence in one aspect the present invention relates to a method for increasing energy content of biogas, said method comprising the steps of:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

b. feeding the bioreactor with biomass,

c. injecting H₂ containing gas into the bioreactor, and

d. collecting the upgraded biogas thus produced, wherein said upgraded biogas has a CH₄ content of at least 90%.

By utilizing the process of the invention comprising the element of co-digesting biomass such as manure with acidic compositions such as acidic waste products, the process is sustained as no inhibition of the process occurs due to increased pH in the bioreactor. Hence, in another aspect the present invention relates to a method for sustained anaerobic conversion of CO₂ to CH₄, said method comprising:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

By lowering the amount of CO₂ of the biogas contained in e.g., a biogas reactor the partial pressures of the biogas components are altered. Accordingly, in one aspect the present invention relates to a method for increasing the partial pressure of CH₄ in biogas, said method comprising:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

Likewise, by increasing the amount of CH₄ of the biogas contained in e.g. a biogas reactor the partial pressure of the biogas components are altered. Accordingly in one aspect the present invention relates to a method for decreasing the partial pressure of CO₂ in biogas, said method comprising:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

c. feeding the bioreactor with biomass,

d. injecting H₂ containing gas into the bioreactor, and

e. collecting the upgraded biogas thus produced, wherein said upgraded biogas has a CH₄ content of at least 90%.

The upgraded biogas so produced may, if the CH₄ content is sufficiently high, be used directly in the natural gas grid infrastructure.

In one aspect, the biogas is further upgraded with the aim of obtaining a gas suitable for use in the natural gas grid infrastructure or the biogas is purified further to obtain substantially pure methane.

In one aspect the present invention relates to the use of the upgraded biogas as vehicle fuel.

In another aspect the present invention relates to the use of the upgraded biogas in a heating power plant such as a district heating power plant.

In another aspect the present invention relates to the use of the upgraded biogas for electricity production.

Ex Situ Biogas Upgrading

To achieve an even higher concentration of CH₄ in the biogas, it may be advantageous to upgrade the biogas produced by the in situ methods described above even further in a separate reactor, i.e. by utilizing an ex situ method.

Hence, it may be advantageous to apply the present methods for upgrading biogas manufactured externally and transported to the bioreactor of the present invention. In one aspect the present invention thus concerns a method of manufacturing an upgraded high CH₄ content biogas, said method comprising the steps of:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

c. feeding the bioreactor with biomass,

d. injecting H₂ containing gas and biogas into the bioreactor, and

e. collecting the upgraded biogas thus produced, wherein said upgraded biogas has a CH₄ content of at least 90%.

In one aspect the present invention relates to a method of manufacturing an upgraded high CH₄ content biogas, wherein said biogas is produced in a first reactor according to the in situ methods of the present invention, followed by a further upgrading of the CH₄ content of the biogas in a second bioreactor (ex situ) according to a method comprising the steps of:

a. initiating an anaerobic digestion process in a first bioreactor comprising:
i. substrate,
ii. anaerobic inoculum comprising anaerobic hydrogenotrophic methanogenic organisms,

b. feeding the first bioreactor with acidic waste having a pH<5,

c. feeding the bioreactor with biomass,

d. injecting H₂ containing gas into the first bioreactor,

e. transferring the biogas produced in the first bioreactor to a second bioreactor, wherein an anaerobic digestion process has been initiated with:
i. nutrients,
ii. anaerobic inoculum comprising anaerobic hydrogenotrophic methanogenic organisms,

f. feeding the second bioreactor with nutrients,

g. injecting H₂ containing gas into the second bioreactor, and

h. collecting the upgraded biogas thus produced, wherein said upgraded biogas has a CH₄ content of at least 95%.

The nutrients may e.g., be a wastewater solution. The skilled person is able to provide a suitable nutrients solution for optimal microbial growth.

In one embodiment the H₂ containing gas is co-injected into the bioreactor together with the biogas. In another embodiment the H₂ containing gas is injected into the bioreactor prior to the injection of the biogas.

In yet another embodiment the H₂ containing gas is injected into the bioreactor subsequently to the injection of the biogas.

The anaerobic inoculum typically contains anaerobic organisms such as anaerobic archaea. Thus, in one embodiment the anaerobic hydrogenotrophic methanogenic organism is contained in anaerobic sludge. In another embodiment the anaerobic hydrogenotrophic methanogenic organism is contained in anaerobic inoculum.

The anaerobic organisms do not need to be supplied with biomass for growth in the second bioreactor. They are capable of growing solely on the supplied nutrients solution and by converting the H₂ and CO₂ into CH₄. The result is an upgraded biogas with a very high CH₄ content.

With the ex situ method it is possible to obtain an upgraded biogas having at least 95% CH₄, for example at least 96% CH₄, such as at least 97% CH₄, for example at least 98% CH₄, such as at least 99% CH₄, for example at least 99.5% CH₄, such as at least 99.6% CH₄, for example at least 99.7% CH₄, such as at least 99.8% CH₄, for example at least 99.9% CH₄ as measured by CH₄ content of the biogas.

In one embodiment, the CH₄ content of the upgraded biogas produced by the methods of the present invention is at least 97.3%.

In one embodiment a gas injection system comprising hollow fibers is used to introduce the H₂ and biogas into the second bioreactor.

Hydrogen Gas

The methods of the present invention utilize H₂ containing gas as one of the substrates in the conversion of CO₂ to CH₄. The H₂ containing gas may be pure H₂ or H₂ in combination with other gases. Preferably the H₂ containing gas is pure H₂. Thus, in one embodiment the H₂ containing gas mentioned herein above consists essentially of H₂ (g).

Hydrogen gas or hydrogen containing gas can be produced by any process known to those skilled in the art. Preferably the H₂ (g) is produced by utilizing excess electric power from a sustainable energy power plant for the hydrolysis of water thus producing H₂ (g). In one embodiment the sustainable energy power plant is a wind power plant.

In another embodiment the H₂ containing gas is obtained from coke oven gas, syngas from biomass gasification and/or waste gas from chemical process industries such as hydrogen from production of potassium chlorate.

Biogas Utilization

Biogas can be utilized for electricity production on sewage works in a CHP gas engine, where the waste heat from the engine is conveniently used for heating the digester; cooking; space heating; water heating; and process heating. If compressed, it can replace compressed natural gas for use in vehicles, where it can fuel an internal combustion engine or fuel cells and is a much more effective displacer of carbon dioxide than the normal use in on-site CHP plants.

Methane within biogas can be concentrated via a biogas upgrader to the same standards as fossil natural gas, which itself has had to go through a cleaning process, and becomes biomethane. If the local gas network allows for this, the producer of the biogas may utilize the local gas distribution networks. Gas must be very clean to reach pipeline quality, and must be of the correct composition for the local distribution network to accept. Carbon dioxide, water, hydrogen sulfide, and particulates must be removed if present.

The requirements to the purity and composition of the biogas for distribution in the natural gas grid infrastructure vary in different countries. For instance, the Danish national requirements to biogas include e.g., a wobbe index in the range of 50.8 to 55.8 MJ/Nm³ and a CO₂ content less than 2.5%. For example, a biogas with a CH₄ content of 97.3% has an acceptable wobbe index and may be used directly in the Danish natural gas grid infrastructure.

In one embodiment, the biogas produced by the present invention is added propane to adjust the burn value (wobbe index) of the gas.

In one aspect the biogas upgraded by the process of the present invention is used as vehicle fuel.

In one embodiment the biogas upgraded by the process of the present invention is used in a heating power plant such as a district heating power plant.

In another embodiment the biogas upgraded by the process of the present invention is used for electricity production.

Fertilizer

As mentioned herein above, the method of the present invention can utilize manure such as cattle manure for co-digestion with acidic waste in the reactor. The digested manure is useful for several industrial and agricultural purposes such as a fertilizer.

Thus in one aspect the present invention relates to a method of manufacturing digested manure, said method comprising:

a. initiating an anaerobic digestion process in a bioreactor comprising:
i. substrate,
ii. anaerobic inoculum comprising anaerobic hydrogenotrophic methanogenic organisms,

b. feeding the bioreactor with acidic waste having a pH<5,

c. feeding the bioreactor with biomass comprising undigested manure,

d. injecting H₂ containing gas into the bioreactor, and

e. collecting the digested manure.

In one embodiment the digested manure is used as a fertilizer. In another embodiment the method further comprises refining the digested manure to commercial grade fertilizer.

Bioreactor of the Invention

In one aspect the present invention concerns a bioreactor comprising:

a. biomass,

b. hydrogenotrophic methanogenic organisms,

c. H₂ containing gas, and

acidic waste.

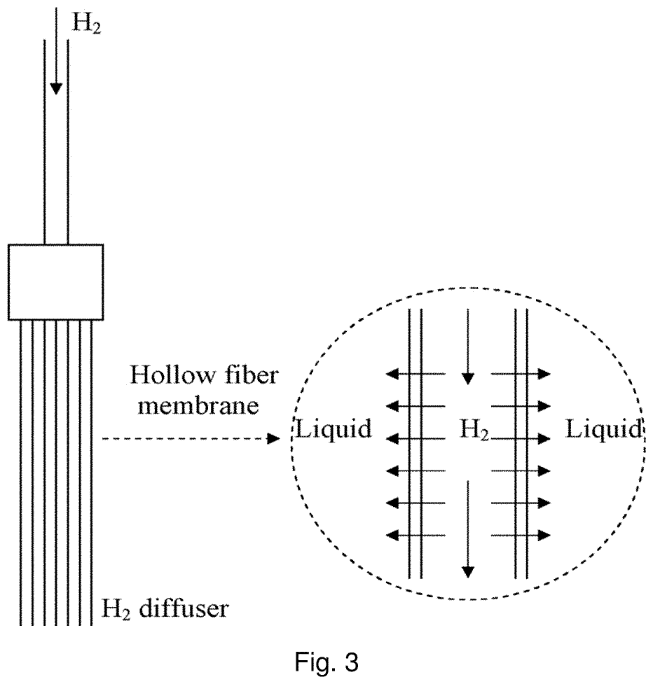

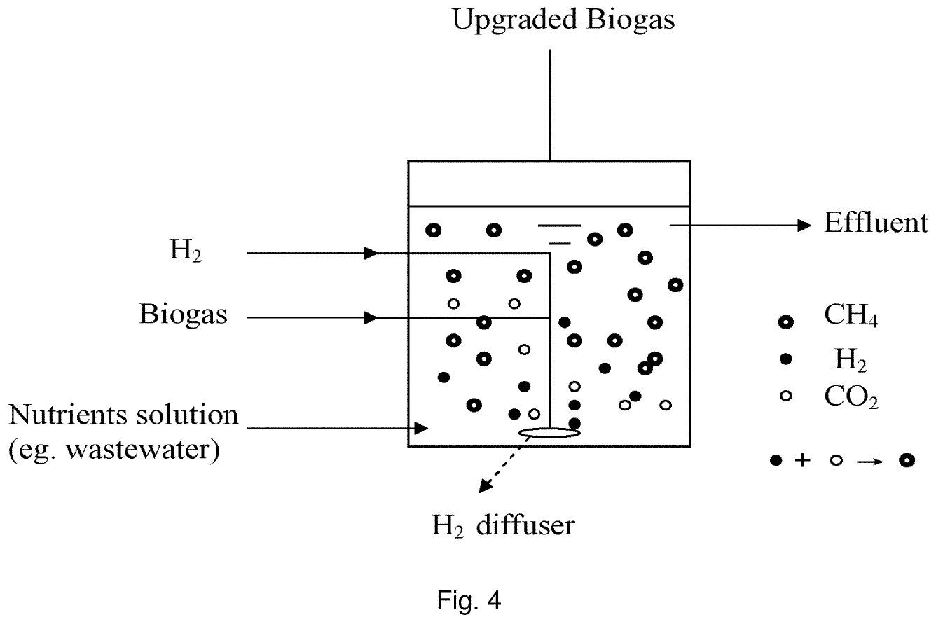

The present inventors have demonstrated that the methods for upgrading biogas according to the present invention is particularly efficient if a bioreactor is used which comprises a particular gas injection system capable of injecting hydrogen containing gas into the fluid phase of the bioreactor content, without causing bubbles. Such an injection system with hollow fiber membranes is illustrated in FIG. 3.

H₂ is a gaseous substrate, and it is not easily captured by the microorganisms in the liquid phase. Full utilization of H₂ is important to further increase the CH₄ content in the biogas. Bubbleless gas-transfer via hollow fiber membrane (HFM) is a potential method to achieve the purpose of complete utilization of H₂. Gas inside the HFM can be delivered into the liquid phase through membranes by diffusion. Thus, the gas is directly dissolved in the liquid, no bubble is formed and the H₂ is taken up by the microorganisms more efficiently.

Hence, in one aspect, the present invention relates to a bioreactor comprising:

a. a reactor vessel,

b. a gas injection system comprising hollow fiber membranes,

c. loading means for supply of substrates and or inoculum, and

d. discharge means for products and/or waste.

The gas injection system comprising hollow fiber membranes is used to introduce H₂ containing gas into the bioreactor.

The gas injection system comprising hollow fiber membranes may also be used to introduce biogas into the bioreactor for ex situ biogas upgrading.

In one aspect the bioreactor comprises biomass, acidic waste and/or nutrients. The biomass, acidic waste and nutrients of the reactor are as defined herein above.

In one aspect the bioreactor comprises anaerobic inoculum comprising hydrogenotrophic methanogenic organisms.

In one aspect the bioreactor comprises H₂ containing gas.

In one aspect the bioreactor comprises acidic waste.

Hydrogenotrophic methanogenic organisms The methods, uses and products of the present invention makes use of hydrogenotrophic methanogenic organisms.

In one embodiment, the hydrogenotrophic methanogenic organisms comprises one or more hydrogenotrophic methanogenic archaea, such as one or more species selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

In one embodiment the hydrogenotrophic methanogenic organism is a substantially pure culture of one hydrogenotrophic methanogenic archaea species.

In one embodiment the conditions of the bioreactor include a temperature of 35° C. to 37° C.

In one embodiment the hydrogenotrophic methanogenic archaea is Methanothermobacter thermoautotroiphicus.

In another embodiment the conditions of the bioreactor include a temperature of 50° C. to 60° C.

In one embodiment the hydrogenotrophic methanogenic archaea is selected from the group consisting of Methanocaldococcus fervens, Methanocaldococcus indicus, Methanocaldococcus infernus, and Methanocaldococcus vulcanius.

In yet another embodiment the conditions of the bioreactor include a temperature of 80° C. to 100° C.

It is an object to provide a method of manufacturing a high CH₄ content biogas, said method comprising the steps of:

a. initiating an anaerobic digestion process in a bioreactor comprising a first composition comprising:
i. substrate,
ii. anaerobic inoculum

b. feeding the bioreactor with a second composition having a pH below that of the first composition,

c. injecting H₂ containing gas into the bioreactor,

d. collecting the upgraded, high content CH₄ content biogas thus produced.

It is also an object to provide a method for increasing CH₄ content in biogas, said method comprising the steps of:

a. initiating an anaerobic digestion process in a bioreactor comprising a first composition comprising:
i. substrate,
ii. anaerobic inoculum comprising anaerobic hydrogenotrophic methanogenic organisms

b. feeding the bioreactor with a second composition having a pH below that of the first composition,

c. injecting H₂ containing gas into the bioreactor,

d. collecting the upgraded biogas thus produced.

It is a further object to provide a method for reducing CO₂ (g) content in biogas, said method comprising the steps of:

a. initiating an anaerobic digestion process in a bioreactor comprising a first composition comprising:
i. substrate,
ii. anaerobic inoculum comprising anaerobic hydrogenotrophic methanogenic organisms,

b. feeding the bioreactor with a second composition having a pH below that of the first composition,

c. injecting H₂ containing gas into the bioreactor,

d. collecting the upgraded biogas thus produced.

It is a still further object to provide a method for increasing energy content of biogas, said method comprising the steps of:

a. initiating an anaerobic digestion process in a bioreactor comprising a first composition comprising:
i. substrate,
ii. anaerobic inoculum comprising anaerobic hydrogenotrophic methanogenic organisms,

b. feeding the bioreactor with a second composition having a pH below that of the first composition,

c. injecting H₂ containing gas into the bioreactor, collecting the upgraded biogas thus produced.

Another object provides a method for sustained anaerobic conversion of CO₂ to CH₄, said method comprising:

a. initiating an anaerobic digestion process in a bioreactor comprising a first composition comprising:
i. substrate,
ii. anaerobic inoculum comprising anaerobic hydrogenotrophic methanogenic organisms

b. feeding the bioreactor with a second composition having a pH below that of the first composition,

c. injecting H₂ containing gas into the bioreactor,

d. collecting the upgraded biogas thus produced.

A further object provides a method for increasing the partial pressure of CH₄ in biogas, said method comprising:

a. initiating an anaerobic digestion process in a bioreactor comprising a first composition comprising:
i. substrate,
ii. anaerobic inoculum comprising anaerobic hydrogenotrophic methanogenic organisms,

b. feeding the bioreactor with a second composition having a pH below that of the first composition,

c. injecting H₂ containing gas into the bioreactor,

d. collecting the upgraded biogas thus produced.

A still further object provides a method for decreasing the partial pressure of CO₂ in biogas, said method comprising:

a. initiating an anaerobic digestion process in a bioreactor comprising a first composition comprising:
i. substrate,
ii. anaerobic inoculum comprising anaerobic hydrogenotrophic methanogenic organisms feeding the bioreactor with a second composition having a pH below that of the first composition,

b. injecting H₂ containing gas into the bioreactor,

c. collecting the upgraded biogas thus produced.

Another object provides a method of manufacturing an upgraded high CH₄ content biogas, said method comprising the steps of:

a. initiating an anaerobic digestion process in a bioreactor comprising:
i. substrate,
ii. anaerobic inoculum comprising anaerobic hydrogenotrophic methanogenic organisms,

b. injecting H₂ containing gas and biogas into the bioreactor, and

c. collecting the upgraded, high CH₄ content biogas thus produced.

The H₂ containing gas may be co-injected into the bioreactor together with the biogas.

The H₂ containing gas may be injected into the bioreactor prior to the injection of the biogas.

The H₂ containing gas may be injected into the bioreactor subsequently to the injection of the biogas.

The substrate may be biomass.

The high CH₄ content biogas may contain at least 50% CH₄, such as at least 60% CH₄, such as at least 70% CH₄, such as at least 75% CH₄, such as at least 76% CH₄, such as at least 77% CH₄, such as at least 78% CH₄, such as at least 79% CH₄, such as at least 80% CH₄, such as at least 81% CH₄, such as at least 82% CH₄, such as at least 83% CH₄, such as at least 84% CH₄, such as at least 85% CH₄, such as at least 86% CH₄, such as at least 87% CH₄, such as at least 88% CH₄, such as at least 89% CH₄, such as at least 90% CH₄, such as at least 91% CH₄, such as at least 92% CH₄, such as at least 93% CH₄, such as at least 94% CH₄, such as at least 95% CH₄, such as at least 96% CH₄, such as at least 97% CH₄, such as at least 98% CH₄, such as at least 99% CH₄, such as at least 99.5% CH₄, as measured by CH₄ content of the biogas.

The H₂ containing gas may consist essentially of H₂ (g).

The H₂ containing gas may be produced by utilizing excess electric power from a sustainable energy power plant for the hydrolysis of water thus producing H₂ (g).

The sustainable energy power plant may be a wind power plant.

The H₂ containing gas may be obtained from coke oven gas, syngas from biomass gasification and/or waste gas from chemical process industries such as hydrogen from production of potassium chlorate.

The second composition may further comprise biomass.

The biomass may be protein-rich waste.

The biomass may be manure.

The second composition may comprise acidic waste.

The acidic waste may be carbohydrate-rich waste selected from the group consisting of whey waste, stillage and fruit juice from potato processing industries.

The pH in the bioreactor may be maintained below 8.5.

The pH in the bioreactor may be maintained below 8.3.

The pH in the bioreactor may be maintained below 8.

The pH may be maintained between 7 and 8.

The pH may be maintained between 7 and 8 by the process of co-digesting biomass with acidic waste.

The ratio of biomass to acidic waste may be experimentally determined to maintain the pH between 7 and 8.

The ratio of biomass to acidic waste may be 3 to 2.

The ratio of biomass to acidic waste may be 1 to 1.

The method may further comprise the step of separating CH₄ (g) from the other components of the upgraded biogas produced, thus producing a substantially pure CH₄ (g). The upgraded biogas and/or the substantially pure CH₄ (g) may be used as vehicle fuel.

The upgraded biogas and/or the substantially pure CH₄ (g) may be used in a heating power plant such as a district heating power plant.

The upgraded biogas and/or the substantially pure CH₄ (g) may be used for electricity production.

It is also an object to provide a method of manufacturing digested manure, said method comprising:

a. initiating an anaerobic digestion process in a bioreactor comprising a first composition comprising:
i. biomass comprising undigested manure,
ii. anaerobic inoculum comprising anaerobic hydrogenotrophic methanogenic organisms

b. feeding the bioreactor with a second composition having a pH below that of the first composition,

c. injecting H₂ containing gas into the bioreactor,

d. collecting digested manure thus produced.

The method may further comprise refining the digested manure to a commercial grade fertilizer.

The digested manure may be as a fertilizer.

It is a further object to provide a bioreactor comprising:

a. a reactor vessel,

b. a gas injection system comprising hollow fibers

c. loading means for supply of starting material, and

d. discharge means for products and/or by-products.

The starting material may be anaerobic inoculum comprising hydrogenotrophic methanogenic organisms, nutrients, acidic waste and/or biomass such as manure.

The gas injection system may be used for supplying H₂ containing gas and/or raw low CH₄ content biogas to the reactor vessel.

The product may be upgraded high CH₄ content biogas.

The hydrogenotrophic methanogenic organisms may be hydrogenotrophic methanogenic archaea.

The hydrogenotrophic methanogenic archaea may comprise one or more species selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

The hydrogenotrophic methanogenic (archaea) organism may be a substantially pure culture of one hydrogenotrophic methanogenic archaea species.

The conditions may include a temperature of 35° C. to 37° C.

The hydrogenotrophic methanogenic archaea may be Methanothermobacter thermoautotroiphicus.

The conditions may include a temperature of 50° C. to 60° C.

The hydrogenotrophic methanogenic archaea may be selected from the group consisting of Methanocaldococcus fervens, Methanocaldococcus indicus, Methanocaldococcus infernus, and Methanocaldococcus vulcanius.

The conditions may include a temperature of 80° C. to 100° C.

The anaerobic hydrogenotrophic methanogenic (archaea) organism is contained in anaerobic sludge.

### EXAMPLES

### Example 1: In Situ Upgrading

Material and Methods

Substrate Characteristics

Substrates used in the experiment were cattle manure from Vegger biogas plant and whey from Vestforsyning A/S, Denmark. The substrates were received in one batch, mixed thoroughly and distributed in 5 L plastic bottles. It was kept at −20° C. for the whole period of experiment. The frozen substrates were thawed and kept at 4° C. for 2-3 days before use. Substrate characteristics were analyzed as illustrated in Table 2. Considering the high organic concentration of whey, it was diluted four times and then mixed with manure at a ratio 2:3.

TABLE 2
Substrate characteristics

| | Cattle | | |
|---|---|---|---|
| Parameters | manure | Whey | Mixture |
| pH | 7.15 ± 0.11 | 4.33 ± 0.13 | 6.85 ± 0.10 |
| TS (g/L) | 30 ± 1.1 | 105 ± 2.1 | 28.5 ± 1.1 |
| VS (g/L) | 24.1 ± 1.2 | 98 ± 2.8 | 25 ± 1.4 |
| COD (g/L) | 40.4 ± 2.3 | 150 ± 5.7 | 40 ± 2.3 |
| TKN (mg/L) | 1092 ± 210 | 460 ± 78 | 701 ± 68 |
| NH₄⁺-N | 540 ± 56 | 89 ± 25 | 330 ± 59 |
| (mg/L) | | | |

Reactor Setup and Operations

Two identical 1 L continuously stirred tank reactors (CST R) (A and B) with working volume of 600 mL were used. Both reactors were filled with inoculum, which was digested manure from Snertinge biogas plant, in Denmark, operating under thermophilic conditions (55° C.). Temperature in the reactors was controlled at 55° C. The HRT was controlled at 15 days. The reactors were fed two times per day. Hydrogen was continuously added to Reactor A, while reactor B was operated as control, without hydrogen injection. The H₂ injection flow rate in reactor A was initially set at 1.5 L/d, and then changed to 1.7 L/d after 20 d operation, which was around 4 times higher than CO₂ production rate in the control reactor (B), corresponding to the stoichiometric ratio of H₂:CO₂ for production of CH₄. The hydrogen gas was injected to the bottom of reactor A either by a column diffuser (pore diameters 0.5-1.0 mm) or ceramic diffuser (14-40 μm). The reactors were mixed by magnetic stirrer, and two different stirring speeds 150 and 300 rpm were tested. Detailed operation parameters could be found in Table 3.

TABLE 3
Performances of the reactors

| | Phase I | | Phase II | | Phase III | |
|---|---|---|---|---|---|---|
| Reactor | A | B | A | B | A | B |
| Mixing | 150 | 150 | 300 | 300 | 150 | 150 |
| speed (rpm) | | | | | | |
| Gas diffuser | N | / | N | / | C | / |
| Biogas | 1429 ± 157 | 874 ± 67 | 1235 ± 147 | 895 ± 45 | 1180 ± 130 | 873 ± 55 |
| prodution rate | | | | | | |
| (mL/(L · d)) | | | | | | |
| Biogas | | | | | | |
| composition (%) | | | | | | |
| CH₄ | 53 ± 3 | 55 ± 3.5 | 68 ± 2.5 | 56 ± 2.8 | 75 ± 3.4 | 56.7 ± 1.5 |
| CO₂ | 13 ± 1.5 | 45 ± 3.1 | 8.8 ± 1.8 | 44 ± 2.9 | 6.6 ± 1.2 | 43.3 ± 1.6 |
| H₂ | 34 ± 3.2 | / | 23.2 ± 2.3 | / | 18.4 ± 2.6 | / |
| CH₄ | 757 ± 43 | 480 ± 31 | 839 ± 31 | 501 ± 25 | 885 ± 37 | 494 ± 22 |
| production rate | | | | | | |
| (mL/L · d) | | | | | | |
| CO₂ | 185 ± 36 | 393 ± 27 | 108 ± 35 | 393 ± 26 | 78 ± 24 | 378 ± 18 |
| production rate | | | | | | |
| (mL/L · d) | | | | | | |
| H₂ | 1185 ± 145 | / | 1384 ± 128 | / | 1452 ± 159 | / |
| consumption rate | | | | | | |
| (mL/L · d) | | | | | | |
| pH | 7.74 ± 0.14 | 7.28 ± 0.12 | 7.84 ± 0.1 | 7.33 ± 0.07 | 7.89 ± 0.12 | 7.31 ± 0.13 |
| Acetate (mM) | 2.1 ± 0.1 | 0.6 ± 0.2 | 2.3 ± 0.2 | 0.5 ± 0.3 | 2.5 ± 0.4 | 0.8 ± 0.3 |
| Propionate | 0.8 ± 0.3 | 0.3 ± 0.1 | 0.7 ± 0.4 | 0.2 ± 0.1 | 0.5 ± 0.5 | 0.2 ± 0.1 |
| (mM) | | | | | | |
| Effluent | 8.9 ± 0.5 | 8.5 ± 0.6 | 7.8 ± 0.7 | 8.3 ± 0.4 | 8.4 ± 0.5 | 8.2 ± 0.8 |
| VS (g/L) | | | | | | |

Pulse Load Tests

After the reactors had reached steady state, organic load pulses were introduced to compare the stability of anaerobic reactor with and without hydrogen addition. The tests were performed by feeding both reactors A and B with whey alone without dilution for two days. After that, the reactors were fed with the mixture again.

Results and Discussion

The performances of both reactors A and B at steady-states were summarized as shown in Table 3. The experiments were carried out for around 150 days. Steady state of each operation condition was obtained after around 2-3 HRT operation when VFA and biogas production had reached relatively stable values.

In the whole operation period, pH in reactor A was around 7.7-7.9, which was higher than that from reactor B (around 7.3). It was consistent with our previous study that addition of hydrogen to the anaerobic reactor would increase the pH. However, pH were always below 8 when co-digested manure with whey, which was suitable for methane production. The results supported our hypothesis that co-digestion of manure and whey could decrease the pH compared with manure alone (pH around 8.3). During Phase I, CH₄ content in reactor A was only 53%, which was slightly lower than that from reactor B. However, the CO₂ content (13%) was much lower than that (45%) from reactor B. The CH₄ production rate of reactor A was around 58% higher compared with reactor B due to the conversion of H₂ and CO₂ to CH₄. In order to increase the hydrogen consumption rate and decrease the H₂ content in the biogas, the stirring speed was increased from 150 rpm to 300 rpm (Phase II). The H₂ consumption rate increased from 1185 to 1384 mL/L·d, which resulted in the increase of CH₄ content of reactor A to 68% and the decrease of H₂ content to 23%. It is obvious the increase of mixing was beneficial for high CH₄ content, which could be mainly due to the lower gas-liquid mass transfer limitations. However, the increase of mixing did not result in significant increase of CH₄ production rate of reactor B. The reason could be that 150 rpm was adequate to achieve homogeneous distribution of substrate, enzymes and microorganisms throughout the reactors. Previous studies also showed that vigorous mixing may disrupt the structure of microbial flocks and disturbs the syntrophic relationships between organisms thereby adversely affecting the reactor performances. In addition, the increase of mixing may significantly increase the operation cost, though it could help to increase the hydrogen consumption rate. Therefore, during Phase III, we decreased the stirring speed back to 150 rpm. Instead of intense mixing, we changed the H₂ distribution equipment from column diffuser to ceramic diffuser, which has smaller pores for better H₂ distribution. The CH₄ content further increased to 75%, which was much higher than that of reactor B. The above results indicated that high CH₄ content could be obtained by the increase of mixing or utilization of efficient H₂ distribution equipment. Utilization of efficient H₂ distribution equipment is recommended, because it is difficult to increase the mixing intensity in actual biogas plants. We calculated the theoretical CH₄ production rate based on the H₂ consumption rate and CH₄ production rate from reactor B at each operation condition, and the theoretical value was very close to the measured value, which indicated that the consumed hydrogen was almost fully converted to CH₄. Table 3 also showed that the addition of hydrogen did not influence the effluent VS concentration. Though there were small amount increase of acetate and propionate in reactor A compared with reactor B, their concentrations were still very low (<2 mM). It could be due to the optimal fermentation pH. The acetate concentration in our previous study were 24 mM and 7 mM in the anaerobic reactor fed with manure alone with and without H₂ addition, which could be due to the high pH (>8.0). It further demonstrated the superiority by co-digestion.

After Phase III, performances of reactors A and B under organic loading shock was tested by fed with whey alone without dilution for two days, and then the mixture was fed to the reactors again. For reactor A, the performance recovered within 5 days, and the pH was always higher than 7.0. However, the organic loading shock had significant effect on the performance of reactor B. There was large amount acetate accumulation, and it could be fully degraded only after around 10 day operation. Meanwhile, the accumulation of propionate was also observed, but it was still in a high level (8 mM) even after around 15 day operation. The pH in the reactor even decreased to lower than 6.5. The above results showed that the reactor with addition of H₂ was much stable and could bear the organic loading shock.

### Example 2: Ex Situ Upgrading

Material and Methods

As sources of inoculum, mesophilic anaerobically digested sewage sludge (Wastewater treatment plant, Lundtofte, Denmark) and thermophilic anaerobically digested manure (Biogas plant, Snertinge, Denmark) were used.

Reactor Setup and Operation

The first experiment was conducted to compare the biogas upgrading potentials by mesophilic and thermophilic enriched mixed cultures. Initially, mixtures (380 mL) of mesophilic or thermophilic sludge and basal anaerobic (BA) medium [Angelidaki et al. (2004) Rev Environ Sci Biotech 3:117-129] with VSS concentration 5 g/L were added in 1140 mL serum bottles. The BA medium contained 10 mM PBS buffer. The bottles were then closed with butyl-rubber stoppers and aluminum crimps and flushed with pure H₂. After that, pure CO₂ (190 mL) were injected into the bottles to achieve the ratio of H₂/CO₂. The bottles were incubated in shakers at 37° C. (bottles inoculated with mesophilic sludge) and 55° C. (bottles inoculated with thermophilic inoculum) with shaking speed 300 rpm. The mixtures (25 mL) were replaced with fresh BA medium every day and at the same time the gases were also refreshed. The experiment lasted around 2 months. All the bottles were performed in duplicates. To determine the kinetics of H₂ consumption, enriched mesophilic and thermophilic cultures were incubated with different concentrations of H₂ in the headspace gas as described.

After one month operation of the first experiment, the second experiment was started under thermophilic condition with continuous feeding of gas. The reactor was 1 L bottle with 600 mL working volume. The reactor was filled with thermophilic inoculum and BA medium. The initial VSS concentration was 5 g/L. The feeding gas was composed of H₂, CH₄, CO₂ with the ratio 60:25:15. The gas was injected to the bottom of the reactor through ceramic gas diffusers. The initial gas flow rate was 3 and then increased gradually. 40 mL mixture in the reactor was replaced with fresh BA medium everyday. The reactor was magnetically stirred at 500 or 800 rpm. Steady-state was defined with the differences of biogas production rate and component less than 10% in the consecutive six days.

Specific Methanogenic Activity (SMA) Tests

Acetoclastic and hydrogenotrophic methanogenic activities of the anaerobic cultures from both mesophilic and thermophilic reactors at the beginning and end of the enrichment were tested. 20 mL fresh samples were added in 68 mL bottles. Acetate (20 5 mM) or H₂/CO₂ (80/20, 1 atm) were supplemented to each bottle. Bottles with fresh samples only, but without substrates, were used as controls. The bottles were incubated in shakers at 37° C. or 55° C. with shaking speed 300 rpm. All the tests were prepared in duplicates.

Results and Discussion

Biogas Upgrading Potentials

Mesophilic and thermophilic mixed cultures were enriched to characterize their biomethanation potentials from H₂ and CO₂. The steady-states were achieved after one month cultivation. The long-term enrichments significantly increased the H₂ and CO₂ consumption rates under both mesophilic and thermophilic conditions. At the beginning of the enrichments, there were still CO₂ left even H₂ was fully consumed which could be due to the bicarbonate in the inoculum, which could react with H₂ and therefore decreased the CO₂ consumption in the gas phase. The CO₂ and H₂ could be almost fully consumed after long-term cultivation. At the end of the enrichment, it took around 4 h to achieve complete conversion of H₂ and CO₂ to CH₄ under thermophilic condition, while it took around 8 h under mesophilic condition.

Initially, both mesophilic and thermophilic cultures had acetoclastic and hydrogenotrophic methanogenic activities (around 10 mL CH₄(gVSS·h)). However, the acetoclastic methanogenic activities could not be detected after long-term cultivation, while the hydrogenotrophic methanogenic activities increased to 198 mL CH₄/(gVSS·h)) under mesophilic condition and 320 mL CH₄(gVSS·h) under thermophilic condition.

The results indicated that hydrogenotrophic methanogens were selectively enriched, and thermophilic conditions should provide high efficiency for biogas upgrading. Though acetate was found in both mesophilic (0.5 mM) and thermophilic (6.6 mM) enriched cultures, it was only equal to 0.14% and 1.9% of the produced CH₄. The acetate could be produced from homoacetogens [Luo et al. (2011) Biotech Bioeng 108:1816-1827], and it needs to be further used by acetoclastic methanogens. Normally, in anaerobic digestion process, homoacetogens cannot compete with hydrogenotrophic methanogens, due to their unfavorable thermodynamic characteristics [Siriwongrungson et al. (2007) Water Res 41: 4204-4210].

Biogas Upgrading Performance of Thermophilic Anaerobic Reactor

The biogas upgrading potentials were then tested in thermophilic continuously stirred tank reactor (CSTR), since the conversion of H₂ and CO₂ into CH₄ was significantly faster under thermophilic conditions compared to mesophilic operation. The operation parameters and results are shown in Table 4.

TABLE 4
Performances of the thermophilic reactor under different operation conditions

| | | 11-43 | 44-73 | 74-96 | 97-135 |
|---|---|---|---|---|---|
| Period (day) | 0-10 | (I) | (II) | (III) | (IV) |
| Gas loading rate | 3 | 6 | 12 | 12 | 24 |
| (L/(L_{reactor} · d)) | | | | | |
| Gas retention time (h) | 8 | 4 | 2 | 2 | 1 |
| Mixing speed (rpm) | 500 | 500 | 500 | 800 | 800 |
| Biogas production rate | 1.4 ± 0.3 | 2.5 ± 0.4 | 5.1 ± 0.6 | 4.9 ± 0.5 | 10.1 ± 1.3 |
| (L/(L_{reactor} · d)) | | | | | |
| Biogas composition | | | | | |
| CH₄ (%) | 93.5 ± 4.4 | 95.4 ± 2.8 | 89.9 ± 4.1 | 94.2 ± 2.8 | 90.8 ± 2.4 |
| CO₂ (%) | 4.2 ± 2.5 | 0.7 ± 0.4 | 2.6 ± 1.5 | 1.9 ± 0.5 | 2.2 ± 1.3 |
| H₂ (%) | 2.3 ± 2.4 | 3.9 ± 0.8 | 7.5 ± 1.2 | 3.9 ± 0.7 | 7 ± 1.8 |
| H₂ consumption rate | 2.9 ± 0.5 | 5.9 ± 0.4 | 11.3 ± 0.7 | 11.6 ± 0.8 | 22.8 ± 2.1 |
| (L/(L_{reactor} · d)) | | | | | |
| CH₄ production rate | 0.9 ± 0.2 | 1.5 ± 0.3 | 2.6 ± 0.5 | 2.7 ± 0.6 | 5.3 ± 1.4 |
| (L/(L_{reactor} · d)) | | | | | |
| Yield CH₄/H₂ | 0.31 | 0.26 | 0.23 | 0.23 | 0.23 |
| Acetate concentration | 5.9 ± 1.2 | 8.8 ± 0.7 | 9.8 ± 1.3 | 10.5 ± 1.1 | 9.4 ± 1.6 |
| (mM) | | | | | |

The inoculum has hydrogenotrophic methanogenic activity, and therefore more than half of the H₂ in the feeding gas was immediately consumed once start-up of the reactor. At day 10, the CH₄ content could be increased from 25% in the feeding gas to around 93% in the upgraded gas. After that, the gas loading rate increased to 6 L/(L·d) and the reactor was operated under this condition until steady-state was achieved. The CH₄ content could be as high as 95.4%, and there were only small amounts of H₂ and CO₂ left. The quality of the upgraded biogas was good enough to be used as natural gas. At day 44, the gas loading rate was further increased to 12 L/(L·d). However, the CH₄ content in the upgraded gas decreased to around 90% even after around one month operation. The gas-liquid mass transfer has been found as the limiting factor for bioconversion of gaseous substrate, especially when the gas (CO, H₂) has low solubility. Therefore, the mixing speed was increased from 500 rpm to 800 rpm at day 74 to lower the gas-liquid mass transfer limitations. The increase of mixing speed resulted in the increase of CH₄ content to around 95%. But there was no significant difference of the H₂ consumption rate between Phase II and III. It seems that intensive mixing of the liquid could reduce the H₂ content in the biogas, and thereby result in higher CH₄ content even under high gas loading rate. This point will be discussed later. After day 97, the gas loading rate increased to 24 Though the decrease of CH₄ content was observed again, it is still around 90%. The above results showed that biogas upgrading could be achieved in thermophilic anaerobic reactor with high gas loading rate.

Theoretically, the ratio of CH₄ production rate to H₂ consumption rate should be 0.25. However, the value was more than 0.31 in the initial first 10 days. It could be explained by the excess CH₄ production from inoculum. After long-term operation, the effect of the inoculum was eliminated. The ratios were close to 0.25 during the steady-states of phase I, II, III and IV, which showed that the consumed H₂ was almost stoichiometrically converted to CH₄. Acetate was also detected as the main metabolite in the liquid phase with the concentration between 5.9-10.5 mM. Acetate as a metabolite in the mixed culture has been demonstrated in the first experiment. During the experiment, pH was relatively stable (around 7.8), which was favorable for methanogens.

Biogas upgrading by anaerobic digestion has not been studied before, and the results from this study showed that CH₄ content in the biogas around 90% or even higher could be achieved under thermophilic condition with gas loading rate up to 24 L/(L·d). In Denmark, there are more than 20 centralized biogas plants and cattle manure is the main substrate. The average production of biogas is 140 m³ per 100 m³ reactor tank. If the biogas upgrading concept proposed in the present study is applied in these biogas plants, an anaerobic reactor for biogas upgrading with volume 1/10 of the biogas reactor is needed. If more efficient gas-liquid mass transfer method is adopted, it is expected that even smaller reactor for biogas upgrading can be used. The economic and environmental analysis should be conducted in the future to make comparisons with the current technologies for biogas upgrading.

### Example 3: Bioreactor Comprising Hollow Fiber Membrane (HFM)

Material and Methods

Experimental Setup

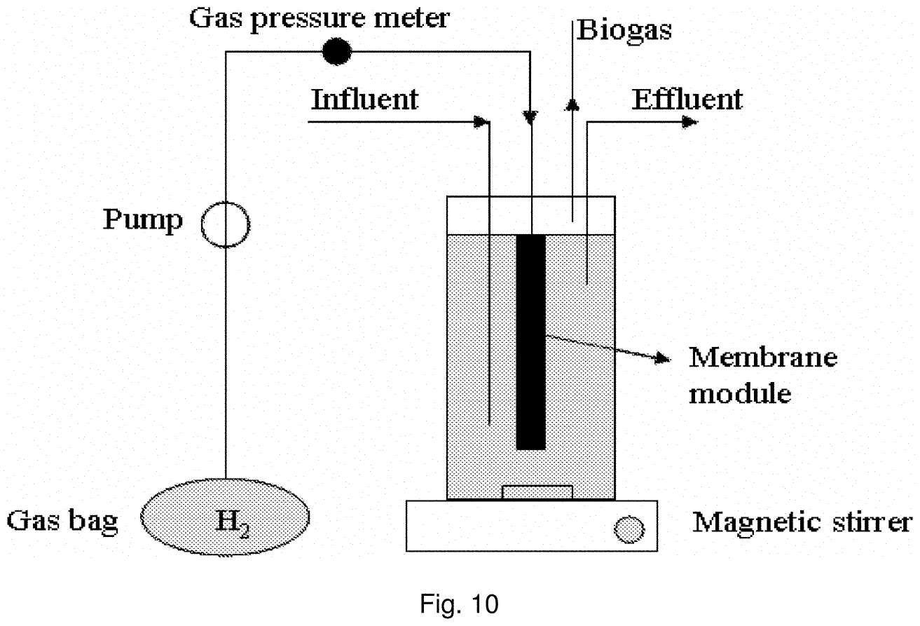

Two identical 1 L continuously stirred tank reactors (CST R) (A and B) with a working volume of 600 mL were used. Before the HFM was installed to reactor A, both reactors were fed with a mixture of cattle manure and whey (TS 28.5 g/L, VS 25 g/L, pH 6.85, detailed information is shown in Table 5) under thermophilic condition (55° C.) with 15 d hydraulic retention time (HRT). The purpose of using co-substrate is to maintain a suitable pH for anaerobic digestion. The reactors were mixed by magnetic stirrer at a stirring speed of 150 rpm and fed once per day. Similar performance was observed in the two reactors after around 1.5 months, and then a HFM module was installed in reactor A. The HFM module contained a bundle of 400 hollow fiber membranes (model MHF 200TL, Mitsubishi Rayon). For the HFM, nonporous thin polyurethane membrane is sandwiched between two porous polyethylene layers. The outside diameter and length of the fiber were 284 μm and 20 cm, respectively, providing a total surface area of 713 cm² for 400 HFM. H₂ was pumped to the HFM module from a gas bag using gas-tight tube. The daily H₂ flow rate was calculated by measuring the initial and residual H₂ inside the gas bag using 100 mL gas-tight syringe. Different H₂ flow rates were obtained by adjusting the speed (2, 3, 4 rpm) of peristaltic pump. The pressure inside the HFM module was monitored by a gas pressure meter. The configuration of reactor A is shown in FIG. 10.

Determination of H₂ Consumption Rate by the Biofilm Formed on the Membrane

During steady-state of each operation period, a similar reactor (C) as reactor A was used to test the H₂ consumption rate by the biofilm formed on the membrane. Reactor C was filled with BA medium (600 mL). NaHCO₃ (5 g/L) was added to the BA medium to provide CO₂ for CH₄ production. Reactor C was then flushed with a gas mixture of (N₂/CO₂, 80/20) and pre-incubated at 55° C. The pH was adjusted to 7.5 by adding IN NaOH or IN HCl. The HFM module was taken out from reactor A, and immediately inserted into reactor C. The reactor was mixed by magnetic stirrer at stirring speeds 150 rpm. A H₂ container was then connected with the HFM module, and the pressure inside the HFM module was controlled at the same level as that in reactor A. Since the bicarbonate in the liquid was consumed due to H₂ addition, increase of pH was expected. Therefore, the experiment only lasted for 4 hours to make sure that the pH inside the reactor was not significantly increased. The produced CH₄ was measured and was used for the calculation of H₂ consumption rate. The experiment was conducted twice at different periods with the same operation conditions, for statistical verification.

Specific Methanogenic Activity (SMA) Tests

Specific methanogenic activity assays on specific substrates during steady-state conditions of both reactors were carried out. 5 mL samples were immediately transferred from the reactors to 20 mL serum bottles. The samples were supplemented with either acetate (20 mM) or H₂/CO₂ (80/20, 1 atm) as substrates. Bottles with reactor samples only, but without substrates, were used as controls. The bottles were incubated in a shaker at 55° C. with shaking speed 300 rpm. All the tests were prepared in duplicates.

Scanning Electron Microscopy Imaging (SEM)

The HFM was prepared for SEM imaging. The sample was first fixed by a sequential two-step chemical immersion for 1 h each, first in 2% glutaraldehyde and then in 1% osmium tetroxide, both at pH 7.2 in 0.1M NaPO4. Samples were dehydrated by successively passaging through 25, 50, 75, 80, 90, 95, 100% ethanol, and then dried by using vacuum drying apparatus. Then the samples were mounted on stubs and sputter-coated with gold-palladium. SEM imaging was carried out with a Quanta Scanning Electron Microscope.

Analytical Methods

TS, VS, NH3-N, TKN and COD were analyzed according to APHA. Total inorganic carbon (TIC) was determined by a TOC-5000 (Shimadzu, Kyoto, Japan). The concentrations of acetate, butyrate, propionate were determined by gas chromatograph (GC) (Hewlett Packard, HP5890 series II) equipped with a flame ionization detector and HP FFAP column (30 m×0.53 mm×1.0 pm). H₂ was analyzed by GC-TCD fitted with a 4.5 m×3 mm s-m stainless column packed with Molsieve SA (10/80). CH₄ was analyzed with GC-TCD fitted with paralled column of 1.1 m× 3/16″ Molsieve 137 and 0.7 m× ¼″ Chromosorb 108.

Calculation of Inorganic Carbonate Species (CO₂ HCO₃)

TIC consists of the three species soluble CO₂, HCO₃⁻ and CO₃²⁻. The fractional ionization factor for carbonic acid (α₀) was calculated using the following equations:

$$\begin{matrix}{α}_{0} = \frac{[ {CO}_{2} ]}{TIC} = \frac{{[ {H}^{+} ]}^{2}}{{[ {H}^{+} ]}^{2} + [ {H}^{+} ] \cdot {K}_{a , 1} + {K}_{a , 1} \cdot {K}_{a , 2}} & ( 2 ) \\ {K}_{a , 1} = \frac{[ {H}^{+} ] [ {HCO}_{3}^{-} ]}{[ {CO}_{2} ]} & ( 3 ) \\ {K}_{a , 2} = \frac{[ {H}^{+} ] [ {CO}_{3}^{2 -} ]}{[ {HCO}_{3}^{-} ]} & ( 4 ) \\ TIC = [ {CO}_{2} ] + [ {HCO}_{3}^{-} ] + [ {CO}_{3}^{2 -} ] & ( 5 )\end{matrix}$$

The Ka values (K_{a,1} is 10^{-6 18} 10^{-10.07}, at 55° C.) was obtained by making temperature corrections to that at 25° C. by Van't Hoff relationship. Based on the above equations, the inorganic carbonate species were calculated.

Results and Discussion

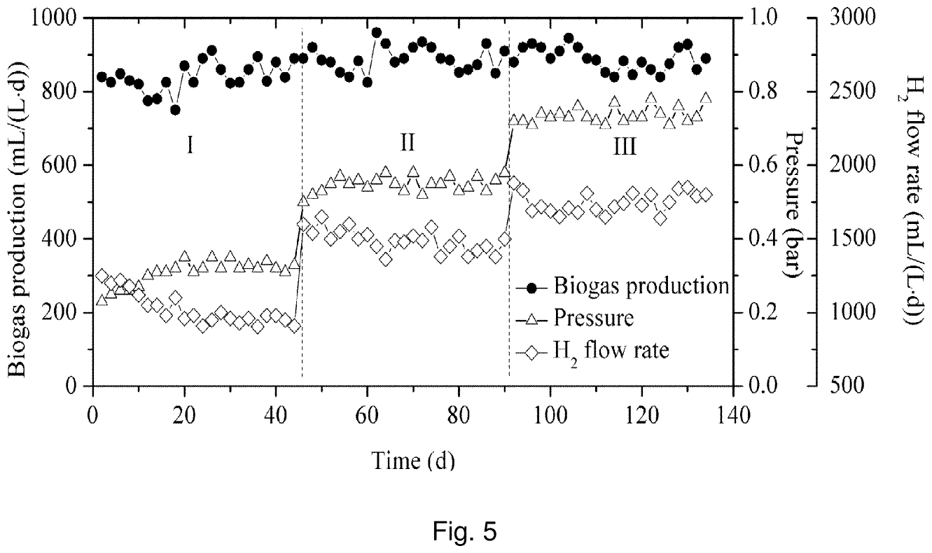

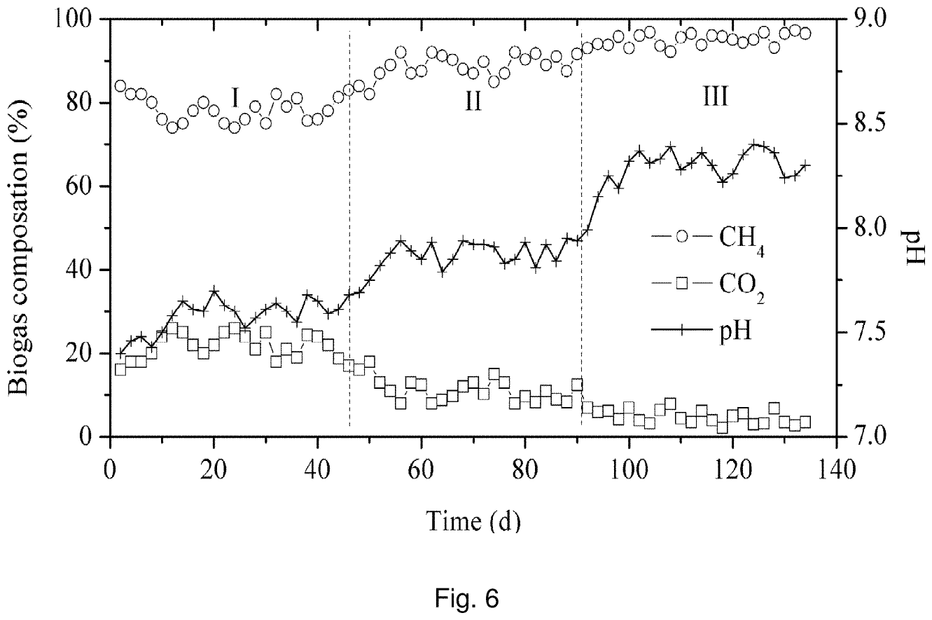

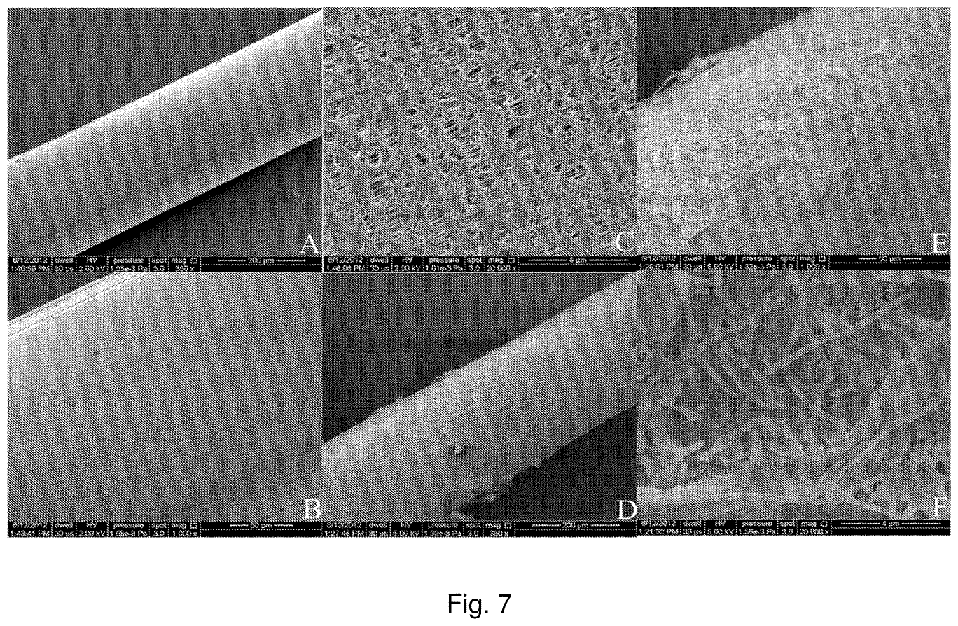

Data from operation of both reactors A and B during steady-states is summarized in Table 5. FIGS. 5 and 6 shows the time-courses of key parameters (biogas production, hydrogen flow rate, biogas composition etc.) in reactor A throughout the study. Reactor B was stable during the whole operation period, and thus the time-course of the measured parameters are not shown. Steady state at each operation condition in reactor A was obtained after operation for approximately 2-3 HRTs, when VFA and biogas production had reached relatively stable values (max. 10% daily variation).

TABLE 5
Summary of steady-state reactor performances

| | Phase I | | Phase II | | Phase III | |
|---|---|---|---|---|---|---|
| | (days 1-45) | | (days 46-91) | | (days 92-134) | |
| Reactor | A | B | A | B | A | B |
| Speed of the pump (rpm) | 2 | / | 3 | / | 4 | / |
| Gas pressure (bar) | 0.32 ± 0.01 | / | 0.56 ± 0.02 | / | 0.75 ± 0.03 | / |
| H₂ flow rate (mL/(L · d)) | 930 ± 50 | | 1440 ± 80 | | 1760 ± 100 | |
| H₂ flow rate | 0.35 | / | 0.54 | / | 0.66 | / |
| (mol H₂/(m² · d)) | | | | | | |
| Biogas production rate | 868 ± 37 | 847 ± 24 | 885 ± 39 | 853 ± 36 | 895 ± 29 | 865 ± 25 |
| (mL/(L · d)) | | | | | | |
| Biogas composition (%) | | | | | | |
| CH₄ | 78.4 ± 1.3 | 54.2 ± 1.6 | 90.2 ± 2.4 | 53.1 ± 1.5 | 96.1 ± 1.1 | 55.4 ± 1.8 |
| CO₂ | 21.6 ± 1.7 | 45.8 ± 1.3 | 9.8 ± 1.3 | 46.9 ± 1.6 | 3.9 ± 1.3 | 44.6 ± 1.9 |
| CH₄ yield (mL/gVS) | 268 ± 24 | 274 ± 16 | 262 ± 31 | 271 ± 19 | 251 ± 22 | 286 ± 17 |
| pH | 7.61 ± 0.06 | 7.29 ± 0.08 | 7.9 ± 0.1 | 7.28 ± 0.11 | 8.31 ± 0.12 | 7.3 ± 0.1 |
| Acetate (mM) | 3.5 ± 0.2 | 1.2 ± 0.3 | 10.2 ± 0.8 | 1.4 ± 0.5 | 36 ± 2.1 | 1.5 ± 0.3 |
| Propionate (mM) | 0.8 ± 0.2 | 0.4 ± 0.1 | 1.6 ± 0.2 | 0.3 ± 0.1 | 1.8 ± 0.4 | 0.4 ± 0.1 |
| Effluent VS (g/L) | 8.3 ± 0.4 | 8.4 ± 0.5 | 8.2 ± 0.5 | 8.4 ± 0.9 | 10.2 ± 0.5 | 8.5 ± 0.6 |
| Total inorganic | 102 ± 8 | 115 ± 11 | 85 ± 5 | 120 ± 12 | 66 ± 7 | 110 ± 11 |
| carbon (mM) | | | | | | |

During the whole operational period, there was no detectable H₂ in the biogas of reactor A. In phase I, the H₂ pumping speed was set at 2 rpm. The CH₄ content in reactor A increased to around 82% once H₂ was fed to the reactor, which indicated that there was high hydrogenotrophic methanogenic activity in reactor A. Obvious increase of H₂ pressure from 0.23 bar to 0.32 bar inside the HFM module was observed during the first 20 days, and it resulted in decrease of H₂ flow rate to the reactor. The H₂ flow rate decreased from around 1200 to 930 mL/L·D). The increase of H₂ pressure may be attributed to biofilm formation on the surface of HFM, which increased the resistance of H₂ diffusion from inside of the hollow fiber to the liquid. A dark grey color, indicating formation of a biofilm, was observed on the surface of HFM. SEM results are shown in FIG. 7. The surface of the raw HFM is porous, however, the porous structure could not be observed on the surface of the HFM at the end of the operation. Biofilm formation is a common phenomenon, and it is very important in a membrane biofilm reactor to retain microorganism in the reactor. However, it seems that biofilm formation is undesirable in our study since there was already sufficient microbial activity in the liquid, while biofilm formation decreased the efficiency of the hydrogen supply.

After 20 days operation, the H₂ pressure was relatively stable at around 0.32 bar, which indicated that a stable biofilm structure was formed on the HFM. During the steady state of phase 1, the H₂ flow rate was 930 mL/(L·d), and the CH₄ content in the biogas was 78%. In order to further increase the CH₄ content, the H₂ pumping speed was adjusted to 3 rpm in phase II. Consequently, the H₂ pressure inside the HFM increased to 0.56 bar and the H₂ flow rate increased to 1440 mL/(L·d). The CH₄ content was increased to around 90% in phase II. Further increase of H₂ pumping speed to 4 rpm in phase III resulted in the even higher CH₄ content (96%) due to the higher H₂ flow rate (1760 mL/(L·d). No further increase of the pumping speed was tested, as the pH in the anaerobic reactor rose to 8.3, which was the upper pH limit for microbial growth. The present study showed that high CH₄ content (>96%) was achieved for H₂ assisted in situ biogas upgrading by HFM.

Besides the CH₄ content, the CH₄ yield is also a key parameter for evaluation of the in situ biogas upgrading process under different operation conditions. Assuming the consumed H₂ was fully converted to CH₄, the CH₄ yield from the organic substrate could be calculated and the data is shown in Table 5. The differences between reactor A and B were lower than 5% in phase I and II. During phase I and II, the pH was always below 8.0. However, the CH₄ yield of reactor A was around 12% lower than that of reactor B in phase III. The lower CH₄ yield of reactor A was consistent with the higher effluent VS and acetate concentration. It suggested that the CH₄ production from the organic wastes was inhibited in phase III in reactor A. It was probably due to the high pH (8.31) in reactor A which inhibited methanogenic activity.

TABLE 6
Specific methanogenic activity on different substrates
(mLCH₄/gVS · d) in reactors A and B at steady-states

| | Phase I | | Phase II | | Phase III | |
|---|---|---|---|---|---|---|
| Reactor | A | B | A | B | A | B |
| Acetate | 123 ± | 117 ± | 116 ± | 125 ± | 90 ± | 131 ± |
| | 27 | 21 | 26 | 18 | 14 | 29 |
| H₂/CO₂ | 295 ± | 210 ± | 330 ± | 201 ± | 352 ± | 221 ± |
| | 25 | 18 | 23 | 29 | 28 | 33 |

From Table 6, it can be seen that the activity of acetoclastic methanogens from reactor A (90 mLCH₄/(gVS·D) was lower than that from reactor B (131 mLCH₄(gVS·d)) in phase III. In the present study, co-substrate of manure and whey was used, and it seems that once we maintained the pH below 8.0 by controlling the H₂ flow rate using HFM, maximum CH₄ content of 90% was achieved without inhibition of the original anaerobic process. If higher CH₄ content is required, it is necessary to increase H₂ flow rate and at the same time control the pH below 8.0 by on-line pH control. In the present study, there was no obvious accumulation of propionate during all the three phases in reactor A, which showed that injection of H₂ by HFM did not inhibit the propionate degradation. It was mainly due to the fast consumption of H₂ by the microorganisms in the reactor, which resulted in the low soluble H₂ concentration and made the degradation of propionate thermodynamically feasible.

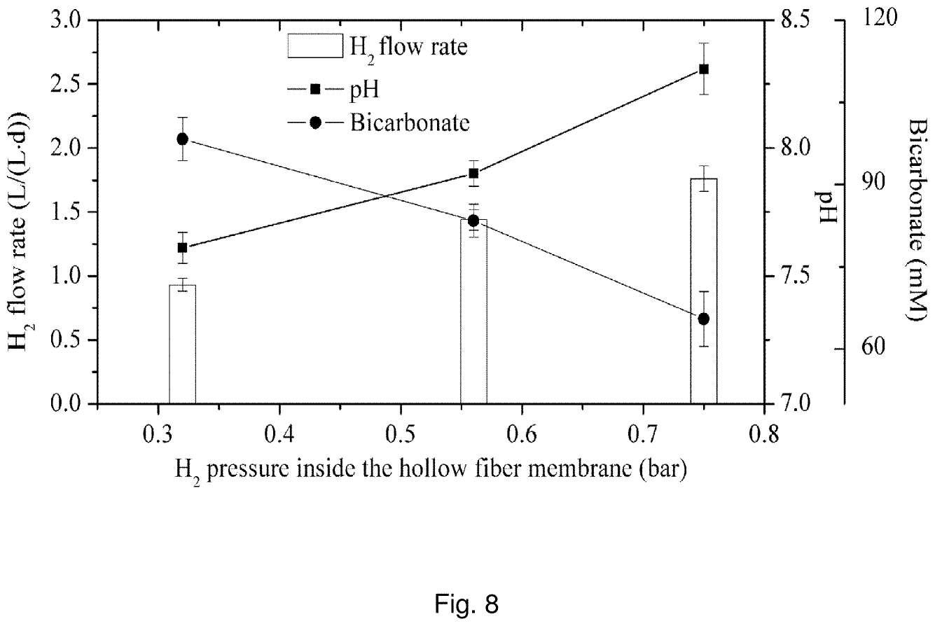

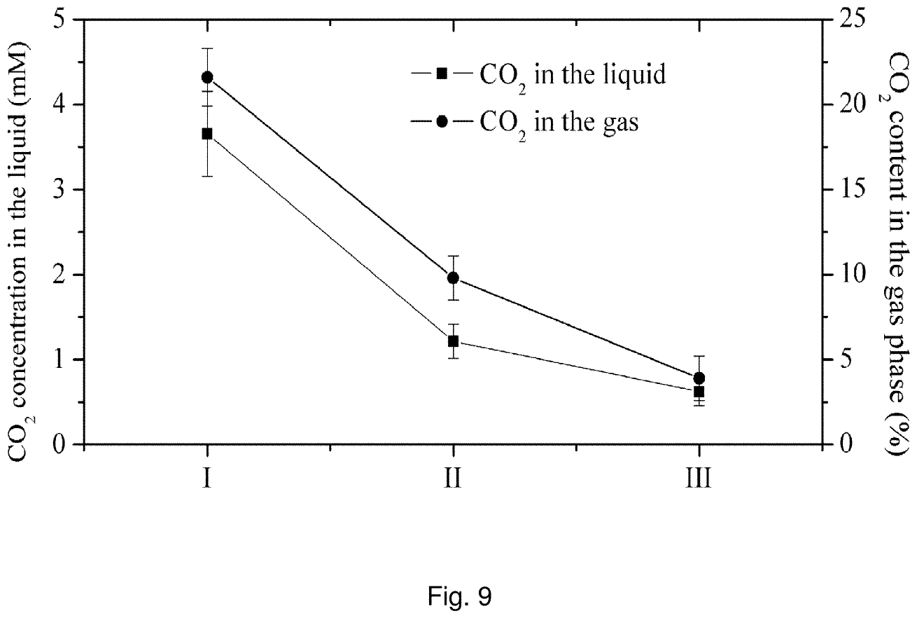

FIG. 8 shows the H₂ pressure, H₂ flow rate, pH and bicarbonate during steady state of each operational period. It is obvious that increase of H₂ pressure was coinciding with increase of flow rate and pH. Based on the inorganic carbon concentration, the soluble CO₂ was calculated and it was shown in FIG. 9. The CO₂ content in the biogas was also plotted in FIG. 9. The decrease of soluble CO₂ from phase I to III was consistent with the decrease of CO₂ content in the biogas. Generally, CO₂ can be assumed to be in thermodynamic equilibrium between liquid and gas phase considering its high solubility. Therefore, Henry's law is used to describe the relationship between soluble and liquid CO₂.

p=k_{H,pc}*x (6)

where P is the partial pressure of CO₂,k_{H,pc} is the Henry's law constant, 1.63 L·atm/mol, x is the soluble CO₂ concentration, mol/L. From equation 6, the CO₂ content in gas phase is proportional to the soluble CO₂ in the liquid, which is consistent with our results. The soluble CO₂ is related to both pH and TIC (equation 2), and the lower concentration of soluble CO₂ can be obtained by either increasing the pH or decreasing the inorganic carbon concentration. In our study, both increase of pH and decrease of inorganic carbon concentration were observed, which finally led to lower CO₂ concentration.

The total H₂ consumption rate and the H₂ consumption rate by the biofilm on the surface of HFM at each operational condition were calculated and listed in Table 7.

TABLE 7
Total H₂ consumption rate and H₂ consumption
rate by the biofilm of reactor A

| | Phase I | Phase II | Phase III |
|---|---|---|---|
| Total H₂ consumption rate (mL/d) | 560 | 864 | 1056 |
| H₂ consumption rate by the biofilm (mL/d) | 200 | 224 | 232 |
| Ratio (%) | 36% | 26% | 22% |

The H₂ consumption rates by the biofilm were 200, 224 and 232 mL/d in phase I, II and III, respectively. However, the H₂ consumption rate by the biofilm only accounted for 36%, 26% and 22% of the total H₂ consumption rate in phase I, II and III, respectively. This indicated that most of the H₂ was consumed by the suspended sludge in the liquid, and it was further proven by the SMA results. In each phase, the SMA of the suspended sludge on H₂ from reactor A was always higher than that from the control reactor B. The above results showed that both the biofilm and suspended sludge were contributing to the H₂ consumption in our HFM based anaerobic reactors. The biofilm increased the resistance of transferred to the liquid, and as a consequence pumping speed and thereby the energy consumption should be increased, in order to maintain constant supply of H₂ to the reactor. The biofilm structure is ultimately a function of the interactive strength between aggregates and hydrodynamic shear force. A previous study of a membrane biofilm reactor for H₂-driven denitrification of municipal wastewater (Celmer et al. 2008. Water Research 42(12)) showed that different biofilm structures (thickness, density) were formed as a result of different mixing intensities. It is obvious that reducing the biofilm thickness by intensive mixing leads to higher gas permeation rate. However, increase of mixing intensity is not practical for anaerobic reactors, due to increase of the operation costs. Additionally, increase of mixing intensity has been proven not to be beneficial for the biogas production. A possible way to remove biofilm is to wash the HFM module periodically.

In our study we successfully applied HFM in an anaerobic reactor for H₂ assisted in-situ biogas upgrading, which solved the problem of low CH₄ content and high H₂ content in the produced biogas. A CH₄ content of 90-96% was achieved in the present study.

What is claimed is:

1. A method of producing a biogas in situ, comprising:
anaerobically digesting a biomass in a bioreactor, with an anaerobic inoculum comprising anaerobic hydrogenotrophic methanogenic organisms, the digestion of the biomass diminishing acid within the bioreactor while producing methane and carbon dioxide;
feeding the bioreactor with an additional biomass over time to maintain the digestion of the biomass and additional biomass in a stead state;
injecting hydrogen gas into the bioreactor, in a sufficient amount to permit the anaerobic hydrogenotrophic methanogenic organisms to reduce at least a portion of the carbon dioxide to methane;
monitoring a pH in the bioreactor; and
adding an acidic waste biomass substrate having a pH less than 5.5 to the bioreactor to maintain the pH in the bioreactor within a range of pH 7 to pH 8, the addition of the acidic waste biomass substrate to the bioreactor lowering a pH of the bioreactor responsive to consumption of acid in the bioreactor by the anaerobic hydrogenotrophic methanogenic organisms,
wherein the bioreactor produces a biogas having at least 90% methane content over a period of time comprising the feeding of the bioreactor with the additional amount of biomass over time.

2. The method of claim 1, wherein the biomass is protein-rich organic waste selected from the group consisting of manure, activated sludge from a wastewater treatment plant, and fish processing residues.

3. The method of claim 1, wherein the biomass and the additional biomass are the same type of biomass.

4. The method of claim 1, wherein the biomass is manure.

5. The method of claim 1, wherein the acidic waste biomass is a carbohydrate-rich waste selected from the group consisting of whey, stillage, fruit juice, and waste from potato processing industries.

6. The method of claim 1, wherein the acidic waste biomass is whey.

7. The method of claim 1, wherein the acidic waste biomass and the additional biomass are fed as a mixture having a pH of less than 7 to the bioreactor at least once per day.

8. The method of claim 1, wherein the additional biomass and the acidic waste biomass are together fed to the bioreactor in a ratio between 3 to 2 and 1 to 1.

9. The method of claim 1, wherein the hydrogen containing gas consists essentially of H₂.

10. The method of claim 1, further comprising:
transferring the biogas produced in the bioreactor to a second bioreactor, wherein an anaerobic digestion process has been initiated with nutrients, and an anaerobic inoculum comprising anaerobic hydrogenotrophic methanogenic organisms;
feeding the second bioreactor with nutrients;
injecting hydrogen containing gas into the second bioreactor;
upgrading the biogas in the second bioreactor; and
collecting the upgraded biogas from the second bioreactor.

11. The method of claim 10, wherein a CH₄ content of the upgraded biogas is at least 95%.

12. The method of claim 1, wherein the anaerobic hydrogenotrophic methanogenic organisms comprise one or more archaea species selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter the rmautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

13. A method of producing a biogas in situ, comprising:
providing a bioreactor having anaerobic hydrogenotrophic methanogenic organisms and an initial substrate to form a biogas within the bioreactor;
injecting hydrogen into the bioreactor, in a sufficient amount to reduce a carbon dioxide portion of the biogas within the bioreactor to methane and causing a corresponding increase in pH;
feeding the bioreactor with a mixture having a pH of less than pH 5.5 of an amount of a biomass and an amount of an acidic waste substrate, a ratio of the acidic waste substrate to the biomass being controlled to maintain the pH in the bioreactor to a pH between 7 and 8 to counteract the corresponding increase in pH; and
collecting the biogas having a methane content of at least 90% derived from the initial substrate, acidic waste substrate, and the biomass.

14. The method of claim 13, further comprising:
transferring the collected biogas to a second bioreactor, wherein an anaerobic digestion process has been initiated with nutrients and an anaerobic inoculum comprising anaerobic hydrogenotrophic methanogenic organisms;
feeding the second bioreactor with nutrients while maintaining a pH between 7 and 8 in the second bioreactor;
injecting hydrogen containing gas into the second bioreactor;
collecting an upgraded biogas from the second bioreactor having a methane content of at least 95%.

15. A system for producing a biogas, comprising:
a bioreactor, configured to anaerobically digest a biomass with an anaerobic inoculum comprising anaerobic hydrogenotrophic methanogenic organisms, the digestion of the biomass diminishing acid within the bioreactor while producing methane and carbon dioxide;
a port configured to receive into the bioreactor over time an additional biomass adapted to maintain the digestion of the biomass and additional biomass in a stead state;
a hydrogen injection system, configured to inject hydrogen gas into the bioreactor, in a sufficient amount to permit the anaerobic hydrogenotrophic methanogenic organisms to reduce at least a portion of the carbon dioxide to methane;
a pH monitor, configured to monitor a pH in the bioreactor; and
a control, receiving feedback from the pH monitor, configured to add an acidic waste biomass substrate having a pH less than 5.5 to the bioreactor to maintain the pH in the bioreactor within a range of pH 7 to pH 8, the addition of the acidic waste biomass substrate to the bioreactor lowering a pH of the bioreactor responsive to consumption of acid in the bioreactor by the anaerobic hydrogenotrophic methanogenic organisms.

16. The system of claim 15, wherein the bioreactor produces a biogas having at least 90% methane content over a period of time comprising the feeding of the bioreactor with the additional amount of biomass over time.

17. The system of claim 15, wherein the hydrogen injection system comprises hollow fibers.

18. The system of claim 15, further comprising a control configured to control a mixing of the acidic waste biomass and the additional biomass to produce a mixture having a pH of less than 7, and to control a feed of the mixture into the bioreactor at least once per day to maintain the pH in the bioreactor below pH 8.

19. The system of claim 18, wherein the acidic biomass has a pH less than 5, and additional biomass and the acidic waste biomass are mixed in a controlled ratio between 3 to 2 and 1 to 1.

20. The system of claim 15, further comprising:
a second bioreactor configured to receive the biogas produced in the bioreactor, the second bioreactor being configured to support an anaerobic digestion process dependent on nutrients and an anaerobic inoculum comprising anaerobic hydrogenotrophic methanogenic organisms;
a second port configured to receive a feed of the nutrients into the second bioreactor;
a second hydrogen injection system configured to inject hydrogen containing gas into the second bioreactor; and
a methane quality detector configured to determine a methane content of an upgraded biogas from the second bioreactor.
